(19) United States
(12) Patent Application Publication
(10) Pub. No.: US 2025/0058117 A1
(43) Pub. Date: Feb. 20, 2025

(54) IMPLANTABLE BIOELECTRONIC DEVICE AND METHOD OF USING SAME

(71) Applicant: Amy Elizabeth Rochford, London (GB)

(72) Inventor: Amy Elizabeth Rochford, London (GB)

(21) Appl. No.: 18/438,796

(22) Filed: Feb. 12, 2024

(30) Foreign Application Priority Data
Aug. 15, 2023 (GB) GB2312476.1

(51) Int. Cl. A61N 1/36; C12M 1/42; C12N 5/074

(52) U.S. Cl. CPC A61N 1/36103; A61N 1/36128; C12M 35/00; C12N 5/0696; C12N 2533/30; C12N 2533/50

(57) ABSTRACT

In general terms, the present invention provides an implantable bioelectronic device, the implantable bioelectronic device comprising a base material with a top layer and a bottom layer opposite the top layer, the base material comprising at least one electrical component. A biological sample seeded on the top layer of the base material. Further, the present invention also provides a method of using an implantable bioelectronic device into a body of a subject, the method comprising performing an in vitro activity for cell culture on the implantable bioelectronic device.

### TECHNICAL FIELD

This invention relates to medicament dispensers for bioelectronic devices. In particular, though not exclusively, this invention relates to an implantable bioelectronic device and to a method of using the implantable bioelectronic device.

### BACKGROUND

Neurological disability affects over 1.3 billion people worldwide, imposing a significant health, economic and social burden. A major hurdle in reversing the effect of injury to the nervous system is the inherent inability of neurons to regenerate and to re-build disrupted neural circuits. Moreover, due to the aforementioned inability of the neurons, the neurons also fail to restore a lost neurological function in a peripheral neural system.

In this regard, implantable neurotechnology (including neural interfaces such as neuroprosthesis) and cell therapy (including cell transplantation) are rapidly developing as potentially effective treatments to restore the lost neurological (biological) function. Notably, the neuroprosthesis aims to bypass the site of injury with implantable electronic devices by connecting directly one part of the nervous system to another (or a prosthetic limb); moreover, the cell transplantation aims to repair the injury site. Conventional implantable electronic devices include, but may not be limited to, epineural cuff electrodes, Utah electrode arrays, flat interface nerve electrode (FINE), longitudinally implanted intrafascicular electrodes (LIFE), transversal intrafascicular multichannel electrodes (TIME), regenerative sieve electrodes, and regenerative microchannel electrodes, that are translated into clinics. However, the conventional neural interfaces only provide symptom-management strategies for progressive diseases, such as that of the nervous system, but translation into the clinic remains limited due to their limited long-term reliability. Moreover, a critical limiting factor of the conventional implantable electronic devices is the resolution with which nerve inputs are mapped onto implants. Notably, this is determined by a variety of factors such as the proximity between electrically active cells and electrodes, as well as the amplitude of their signals. Moreover, the implantable electronic devices alone lack selectivity and specificity of subpopulations of neurons to record or elicit action potentials to or from the damaged neurons due to an imperfect interface between the implanted electronic device and native tissues. Furthermore, both strategies alone have shown limited efficacy due to several challenges, such as lack of cell guidance (that results in a struggle to re-establish functional connections in existing circuits) and survival after implantation and a foreign body reaction (FBR) that generates a dense scar tissue (or collagen layer) around the electronic interface, preventing the electronic signals reaching the target nerve tissue thereby impairing working thereof. Consequently, the electronic device needs to be replaced with a new one until the FBR process repeats itself. Additionally, Wallerian degeneration is also an issue in amputee patients where the damaged peripheral nerve fibres at the proximal stump die back up towards the torso of the subject. In such cases, even if an electronic device is implanted at the proximal nerve stump, the living nerves continue to die moving further away from the bioelectronic device deeming it useless over time.

There remains a need for improved implantable devices that can enhance the functional neurological restoration. It is an object of the invention to address at least one of the above problems, or another problem associated with the prior art.

### SUMMARY OF THE INVENTION

A first aspect of the invention provides an implantable bioelectronic device, the implantable bioelectronic device comprising a base material with a Young's modulus measurement of stiffness no greater than 1 GPa with a top layer and a bottom layer opposite the top layer, the base material comprising at least one electrical component; a biological sample seeded on the top layer of the base material; and, wherein the implantable bioelectronic device, when in use in vitro, enables the biological sample to grow on the top layer of the base material, and wherein the implantable bioelectronic device, when in use in vivo, enables connecting a first element and a second element for restoration of an interrupted biological function between the first and second elements.

It has been found that such an arrangement may advantageously provide for a combinational biohybrid approach of implantable electronics and living cells for functional neurological restoration that could address these issues. In particular, such an arrangement has been found to advantageously allow for a ‘controllable’ synaptic integration between implanted cells and existing circuitry. In this regard, devices, which can be flexible are combined with various soft materials with stem cell-supportive capacity to enable connecting bioelectronics to human physiology, and also regenerating human physiology with medical devices. In essence, such an arrangement is a way of ‘plugging into’ the nervous system that serves as a living interface that allows the connection of living nerves at one end, to electrical components at another in a subject at a desired location, such as, for example, at a human nerve injury site or in an animal (such as a rat) nerve injury model. Attributes to such a biohybrid implant are: an ability to host and interact with human stem cell-derived cells; the promotion of organised functional cellular integration with living tissue; the reduction of scar tissue (FBR) formation; and the restoration of any lost biological function. This technology aims to shift this paradigm to move towards curative solutions by combining the latest technology in tissue engineering with flexible bioelectronics. Additionally, the disclosed implantable bioelectronic device may allow a biohybrid bridge to link severed human nerves to a smart prosthetic device that allows intuitive control close to that of a natural hand, for example. Moreover, it has been found that in such an arrangement, it may be possible to modify the geometry of the implantable bioelectronic device and the type of biological sample to be grown thereon independently based on the subject and the desired location. Beneficially, the implantable bioelectronic device provides improved electrophysiology recordings from the implantable bioelectronic device compared to conventional thin film electronics alone. Additionally, the disclosed implantable bioelectronic device allows for chronic implantations and the survival of the human-based biological sample inside for example a rat subject.

In some embodiments, the implantable bioelectronic device comprises a base material which has a Young's modulus measurement of stiffness no greater than 1 GPa, preferably no more than 0.8 GPa and more preferably no more than 0.6 GPa. The Young's modulus measurement is a quantification of the impact of stress and strain on a given material under tension or compression. The Young's modulus measurement of stiffness as defined in this embodiment calculates the minimum degree of movement or flexibility following the application of a localised pressure before the material breaks.

Suitably, the base material is a thin flexible film biohybrid that mimics the stiffness of the host or target tissue or organ. Optionally, the target tissue is nervous system tissue such as the nerve or an organ such as the brain. Herein the top layer lays on top of the bottom layer of a planar thin film, and the top layer serves as an electrically active surface that comprises the electronic circuit of the implantable bioelectronic device. Optionally, the electronic circuit comprises at least one electrical component and insulator. Beneficially, more than one electrical component may be used for high selectivity. Optionally the electrical component includes, but is not limited to LEDs and transistors. Optionally, the electrical component is at least one electrode wherein the electrode is a gold electrode. Optionally, each electrode has an area of 50 μm×50 μm with 400 μm spacing between adjacent electrodes on the base material. Suitably, the electrode is coated with a poly(3,4-ethylenedioxythiophene) polystyrene sulfonate (PEDOT:PSS) conducting polymer.

It will be appreciated that the base material is designed to mimic the stiffness of the host or target tissue or organ, allowing the full range of motion of said host or target or organ tissue without impacting the biological function or necessary movements required to maintain the biological functions of the host or target tissue or organ and the subject. Suitably, the base material may be fabricated from a parylene derivative to mimic the stiffness of host or target tissue or organ, for example the host or target nerve or brain.

Optionally, in the implantable bioelectronic device, the biological sample may be seeded on a hydrogel which includes at least one of: a fibrin hydrogel, a poly(ethylene glycol) (PEG) hydroges, a poly(acrylic acid) (PAA) hydrogel, an alginate hydrogel, a chitosan hydrogel, a gelatin-based hydrogel. Optionally, in the implantable bioelectronic device, the biological sample may be seeded on a platform of proteins which includes extracellular matrix proteins.

Suitably, the implantable bioeletronic device comprises holes, wherein the holes are configured for potential vasculature penetration through the implantable bioelectronic device to restore an impaired or lost biological function by allowing intuitive control to mimic the natural control mechanism of the body. Moreover, the vascularisation allows for the diffusion of nutrients, oxygen perfusion and waste products removal to aid implanted cell survival. Optionally, the impaired or lost biological function may be impaired signal transduction. Optionally, the holes occupy an area in a range of 50 μm to 200 μm and surround at least one electrode.

Optionally, the biological sample may be human induced pluripotent stem cells (iPSC) that can be differentiated into cell types including neural and muscle. Beneficially, by seeding the biological sample on a hydrogel on the implantable bioelectronic device, the subject's immune system is not adversely reactive towards the implantable bioelectronic device thereby avoiding the FBR formation at implantation sites. It will be appreciated that the use of a scalable cell source, which can be integrated into the implantable bioelectronic device as a biological target for peripheral nerve inputs for example, may allow for the recording from selected subsets of nerve fibres, decrease axon-electrode distance, and improve signal amplitude, potentially increasing spatial and neuron class recording resolution.

Suitably, the hydrogel is configured for coating the biological sample on the base material. Optionally, the hydrogel is a biodegradable hydrogel. Beneficially, the biodegradable hydrogel protects the structure and function of the biological sample upon implantation of the implantable bioelectronic device into the subject. Additionally, beneficially, the biological sample and the biodegradable hydrogel layers improve the overall biocompatibility of the implantable bioelectronic device.

Biodegradable hydrogel materials that may be used in implantable bioelectronic devices of the present disclosure, for example in flexible implantable bioelectronic devices, optionally include one or more of:
(i) Poly(ethylene glycol) (PEG): PEG hydrogels provide good biocompatibility, low toxicity, and may be tuned to exhibit various mechanical properties depending on a degree of crosslinking within the PEG hydrogels;
(ii) Poly(acrylic acid) (PAA): PAA hydrogels may swell and shrink in response to changes in pH, making them useful for pH-sensitive drug delivery and biosensing applications;
(iii) Alginate: Alginate hydrogels are derived from seaweed and exhibit good biocompatibility, low toxicity, and may be used for cell encapsulation and tissue engineering applications;
(iv) Chitosan: Chitosan hydrogels are derived from chitin, which is a natural polymer found in crustacean shells. Chitosan hydrogels exhibit good biocompatibility, antibacterial properties, and may be used for wound healing and drug delivery applications; and
(v) Gelatin-based hydrogels: Gelatin-based hydrogels are derived from collagen and exhibit good biocompatibility and mechanical properties. The gelatin-based hydrogels may be used for cell encapsulation, tissue engineering, and drug delivery applications.

Aforesaid hydrogels may exhibit unique properties and advantages when used in implantable bioelectronic devices, for example in implantable bioelectronic devices; such unique properties include biocompatibility, responsiveness to external stimuli, and a characteristic in use to mimic biological tissues. Moreover, the hydrogels may be used alone or in combination with other materials to create multifunctional and highly adaptable implantable bioelectronic devices.

It will be appreciated that when in use in vivo, the implantable bioelectronic device may be interfaced with a damaged nerve tissue, or at least partially transected host nerve and/or a nervous system tissue. In this regard, the implantable bioelectronic device may be interfaced with the nervous system tissue such as a nerve, the brain or the spinal cord.

In some embodiments, the base material may further comprise at least one of: a chip, at least one sensor (such as a biosensor, an optical sensor, a chemical sensor, and so on), at least one stimulator (such as an optical stimulator, a chemical stimulator, and so on).

In some embodiments, the restoration of the interrupted biological function between the first element and the second element is recorded as stimulation data by the implantable bioelectronic device. Herein, the stimulation data are action potentials resulting from the host nerves in their native state or upon restoration of the interrupted biological function. It will be appreciated that the implantable bioelectronic device enables efficient electrical recording in vivo. It will be appreciated that the stimulation is not always needed; it is needed only for the initial in vivo characterisation of the implantable bioelectronic device. Notably, herein, the implantable bioelectronic device acts as a recording device. However, in the future, this technology could also be used to stimulate host nerve or brain tissue and/or to record the same with or without stimulation. It will be appreciated that, in freely moving subjects, there is no need to send a stimulation and then record the stimulation data. In the freely moving subject, the implantable bioelectronic device can just record live action potentials from the said subject.

In some embodiments, the electrical stimulation is provided as a pulse of an activation threshold ranging from 10 microampere (μA) to 200 μA (namely, in a range of 10 μA to 200 μA) using a pre-defined duration pulse. Notably, the nerves have a lower activation threshold. Optionally, the pulse may be of 100 μA amplitude. It will be appreciated that the pulses are optimised for different subjects, such as a rat, a human, and so on.

In some embodiments, the number of electrical components is at least two, and wherein the two electrical components are arranged in a symmetrical array occupying an area in a range of 1.0 millimetre (mm)×1.0 mm to 10 mm×10 mm within the base material. Optionally when the electrical component are electrodes, the area of the two electrodes, such as in a multielectrode array (MEA) may be equal to or more than the diameter of the nerve bundle (i.e. 1 mm) to allow higher selectivity of the implantable bioelectronic device. Notably, the area of the implantable bioelectronic device will vary depending on the nerve that the implantable bioelectronic device is interfaced with. Optionally, the two electrodes are arranged in a symmetrical array occupying an area of 2 mm×2 mm. However, it will be appreciated that such an area may be optimized for different subjects, such as a rat, a human, and so on. Notably, an area of 2 mm×2 mm may be specific to the rat sciatic nerve, but can easily be adapted to different nerve sizes, i.e. the area would need to be made larger or smaller for human nerves.

Suitably, the electrical components may be implemented as multiple independent electrical components for high selectivity. Optionally, when the electrical components are electrodes, the electrodes may be 32 in number and may be arranged in an array.

In some embodiments of the present disclosure, the base material comprises a polymer layer, selected from a parylene derivative, deposited on a flexible wafer, selected from a silicon, a glass, or polymers.

In some embodiments, the biological sample is selected from an undifferentiated biological cell type such as human induced pluripotent stem cells (iPSC).

In some embodiments, the first element is an electrically active cell and the second element is selected from an electrically active cell, muscle tissue and an electrical component. Optionally, the first element may be a living nerve, or a damaged nerve's distal or proximal end. Optionally, the second element may be a muscle cell or an electronic component such as a prosthetic limb, a prosthetic arm, a prosthetic foot, a prosthetic ear, and so forth. It will be appreciated that a lost (or impaired or interrupted) biological function between the first element and the second element may be restored using the implantable bioelectronic device therebetween.

In some embodiments, the implantable bioelectronic device further comprises a processing arrangement for processing and analysing the recorded stimulation data; a memory unit; a transmitter that can translate the stimulation data, and a battery unit. Optionally, the transmitter is a short-range RF (radio frequency) transmitter. Optionally, the battery unit is a small internal battery, such as a coin battery. Optionally, the implantable bioelectronic device may comprise a sensor arrangement, an amplifier, and so on.

A second aspect of the invention provides a method of (namely, a method for) using an implantable bioelectronic device, the method comprising performing an in vitro activity for cell culture on the implantable bioelectronic device, the in vitro activity comprising obtaining the implantable bioelectronic device, seeding a biological sample on top of the implantable bioelectronic device and allowing the biological sample to grow for a pre-defined time; and performing an in vivo activity comprising implanting the implantable bioelectronic device with the biological sample thereon into a subject at a desired location, wherein the implantation of the implantable bioelectronic device enables connecting a first element and a second element for restoration of an interrupted biological function between the first and second elements.

Suitably, the pre-defined time of growth of a biological sample in vitro is dependent on a specific cell culture. Notably, different cell types have different culture times to reach a desired growth. It will be appreciated that, in this regard, the biological sample is adhered onto the base material. Moreover, a temporary set-up may be designed to enable culturing of the biological sample on the implantable bioelectronic device before the delicate removal thereof and subsequent implantation thereof into the subject. The temporary set-up may host the implantable bioelectronic device for 10-30 days (namely, in a range of 10 to 30 days) whilst performing human cell culture. The temporary set-up may host the implantable bioelectronic device for 10 days whilst performing human cell culture, such as on human iPSC-derived myocytes, thereon. It will be appreciated that the time period for hosting (namely, hosting period) the implantable bioelectronic device in the temporary set-up may vary based on the cell type desired to be cultured on the implantable bioelectronic device. In this regard, optionally, the temporary set-up may be of cell culture plates comprising polydimethylsiloxane (PDMS). Optionally, the temporary set-up may be implemented as a PDMS well and is removed before surgical implantation of the implantable bioelectronic device.

Optionally, the desired location of implantation of the implantable bioelectronic device in the subject may be a sensorimotor nerve. Optionally, the desired location of implantation of the implantable bioelectronic device in the subject may be the brain.

In some embodiments, the implantable bioelectronic device is, according to the aforementioned, an implantable bioelectronic device.

In some embodiments, the implantable bioelectronic device is implanted in the subject such that a bottom layer of the implantable bioelectronic device is laid against a first part of the subject's body and a top layer with the biological sample faces an electrically active cell proximal to the first part of the subject's body. In an example, the implantation is done into the proximal nerve stump, in such case, the implantable bioelectronic device is laid on the dermis of the subject's body and the top layer with the biological sample faces the electrically active cell, such as the nerve tissue. In another example, the implantation is done into the distal nerve stump, in such case, the implantable bioelectronic device is laid between a severed nerve, or on the surface of the brain.

In some embodiments, the method further comprises recording a stimulation data, in vivo, by the implantable bioelectronic device. In some embodiments, the method may comprise the provision of electrical stimulation by the implantable bioelectronic device.

In some embodiments, the electrical stimulation is provided as a pulse of an activation threshold ranging from 10 μA to 200 μA (namely, in a range of 10 μA to 200 μA) using a pre-defined duration pulse.

In some embodiments, a number of electrical components is at least two, and wherein the electrical components are arranged in a symmetrical array occupying an area in a range of 1.0 mm×1.0 mm to 10 mm×10 mm within the base material. It will be appreciated that thousands of electrical components may be arranged to occupy an area of for example 0.5 centimetres (cm)×0.5 cm when for example the brain is the target area.

In some embodiments, the method further comprises processing and analysing, using a processing arrangement, the recorded stimulation data; storing, in a memory unit, the recorded stimulation data; translating, using a transmitter, the recorded stimulation data, and powering, using a battery unit, the implantable bioelectronic device.

In some embodiments, the method further comprises preparing the implantable bioelectronic device using at least one of: a photolithography technique, a printing technique, a metal lift-off technique. Optionally, the printing technique is a three-dimensional bioprinting (technique).

A third aspect of the invention provides a computer program product comprising a non-transitory machine-readable data storage medium with stored thereon program instructions which when accessed by a processing arrangement cause the processing arrangement to carry out the aforementioned method.

Throughout the description and claims of this specification, the words “comprise” and “contain” and variations of the words, for example “comprising” and “comprises”, mean “including but not limited to”, and do not exclude other components, integers or steps. Moreover, the singular encompasses the plural unless the context otherwise requires: in particular, where the indefinite article is used, the specification is to be understood as contemplating plurality as well as singularity, unless the context requires otherwise.

Preferred features of each aspect of the invention may be as described in connection with any of the other aspects. Within the scope of this application, it is expressly intended that the various aspects, embodiments, examples and alternatives set out in the preceding paragraphs, in the claims and/or in the following description and drawings, and in particular the individual features thereof, may be taken independently or in any combination. That is, all embodiments and/or features of any embodiment can be combined in any way and/or can be in combination, unless such features are incompatible.

### BRIEF DESCRIPTION OF THE DRAWINGS

One or more embodiments of the invention will now be described, by way of example only, with reference to the accompanying drawings, in which:

FIG. 1A is an illustration of an implantable bioelectronic device, in accordance with an embodiment of the invention;

FIGS. 1B and 1C are enlarged views of the implantable bioelectronic device of FIG. 1A;

FIG. 1D is an illustration of the preparation of the implantable bioelectronic device of FIG. 1A for cell culture;

FIG. 1E is a graphical representation of the electrode impedance for a control device and the implanted implantable bioelectronic device of FIG. 1A at different weeks of implantation;

FIG. 1F is an illustration of an experimental timeline showing the transfer of the implantable bioelectronic device of FIG. 1A from an in vitro step to an in vivo step;

FIGS. 2A and 2B are illustrations of the implantation of a control device and the implantable bioelectronic device of FIG. 1A, respectively, in accordance with an embodiment of the invention;

FIGS. 2C and 2D are graphical representations of the biological sample stain intensity with respect to the distance thereof from the implantation site of the control device and the implantable bioelectronic device of FIG. 1A, respectively;

FIGS. 2E and 2F show images of immunofluorescence stains of the control device and the implantable bioelectronic device of FIG. 1A;

FIG. 2G shows the quantification of the neuromuscular junctions (NMJ) density in a subject;

FIG. 3 is a schematic illustration of an implantation of an implantable bioelectronic device, in accordance with an embodiment of the invention;

FIG. 4A are representations of the response of a stimulated nerve to the implantable bioelectronic device and a control device in comparison to a naïve nerve hook electrode;

FIG. 4B is a representation of an average peak-to-peak compound action potential (CAP) amplitude recorded by the electrode of the implantable bioelectronic device, the control device and the naïve nerve hook electrode of FIG. 4A;

FIG. 4C is a graphical representation of peak-to-peak compound action potential (CAP) amplitude recorded by the 28-day implanted implantable bioelectronic device and the control device of FIG. 4A;

FIGS. 5A and 5B are graphical representations of nerve signal traces recorded by the chronically implantable bioelectronic device of FIG. 4A over 4 weeks post-implantation and a signal-to-noise ratio (SNR) of the recorded traces, respectively;

FIG. 6A is a graphical representation of root mean square (RMS) time traces from 3 bipolar electrodes recorded by the implantable bioelectronic device of FIG. 4A over 4 weeks post-implantation;

FIG. 6B is a graphical representation of the quantification of the correlation between the recorded RMS time traces of FIG. 6A and stepping events;

FIG. 6C is a representation of the time-frequency spectrogram and the time trace of a recording from a bipolar electrode of FIG. 6A at week 4 post-implantation;

FIG. 7 is an illustration of the survival of the biological sample 7 days post-implantation of the implantable bioelectronic device;

FIG. 8 is a graphical representation of the cell nuclei stain intensity over a distance from 28 day-implanted implantable bioelectronic device and a control device;

FIG. 9 is a graphical representation of peak-to-peak compound action potential (CAP) amplitude recorded by the 28-day implanted implantable bioelectronic device of FIG. 1A and a control device for 0.1 ms pulses of a range of amplitudes;

FIG. 10 is graphical representation of the quantification of the electrode impedance of the implanted implantable bioelectronic device of FIG. 1A over 4 weeks of implantation;

FIG. 11 is a schematic illustration of the step-by-step process involved in culturing the biological sample onto the implantable bioelectronic device, in accordance with an embodiment of the invention; and

FIG. 12 is a flowchart of steps of a method of using an implantable bioelectronic device, in accordance with an embodiment of the invention.

### DETAILED DESCRIPTION OF THE DRAWINGS

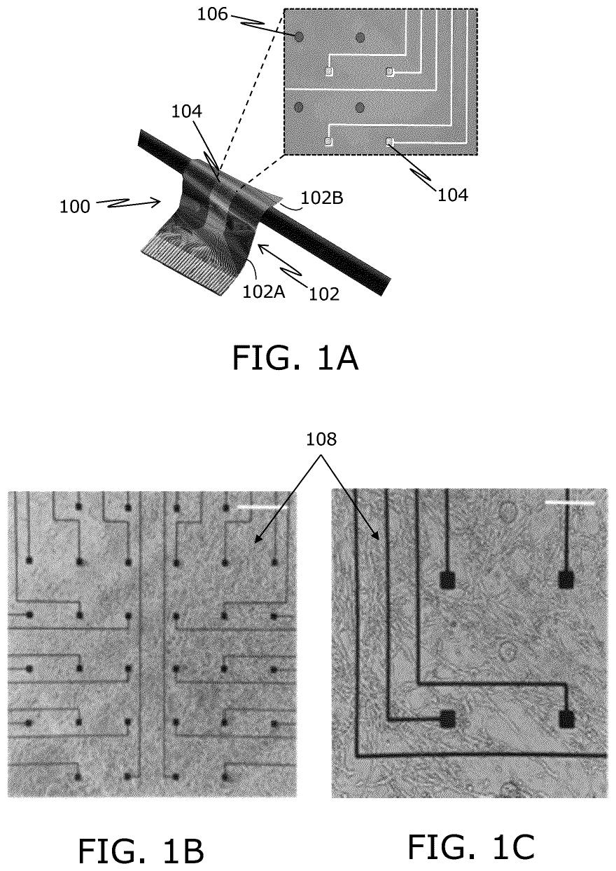

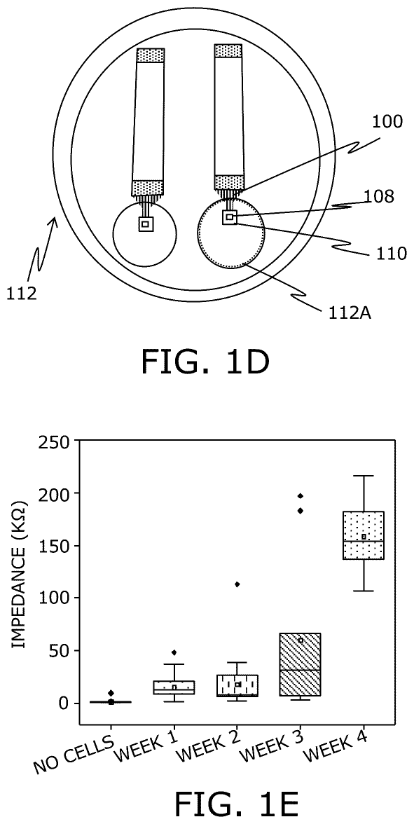

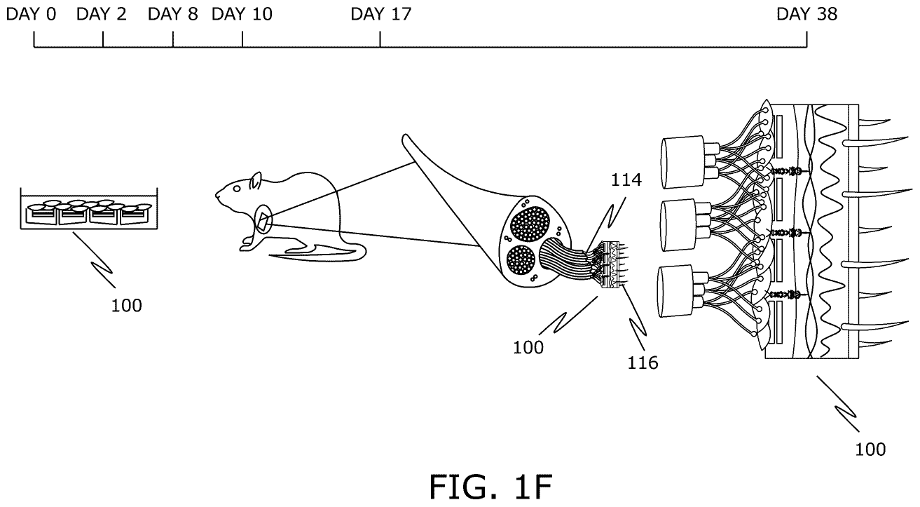

Referring to FIG. 1A, there is shown an illustration of an implantable bioelectronic device 100, in accordance with an embodiment of the invention. The implantable bioelectronic device 100 comprises a base material 102 with a top layer 102A and a bottom layer 102B opposite the top layer 102A, the base material 102 comprising at least one electrical component, wherein the electrical component is at least one electrode 104 and at least one hole 106; and a biological sample (not shown) seeded on a hydrogel, wherein the hydrogel is a biodegradable hydrogel (not shown) on the top layer 102A of the base material 102, wherein the implantable bioelectronic device 100, when in use in vitro, enables the biological sample to grow on the top layer 102A of the base material 102 prior to a coating thereof with the biodegradable hydrogel, and wherein the implantable bioelectronic device 100, when in use in vivo, enables connecting a first element 114 and a second element 116 for restoration of an interrupted biological function between the first element 114 and second element 116. It may be appreciated that the top layer 102A and the bottom layer 102B are two faces of the base material 102. The bottom layer 102B refers to a side of the base material 102 on top of which lies the top layer 102A that comprises multiple electrical components, such as the electrode 104 and at least one hole 106, as shown along with the biological sample and the biodegradable hydrogel. Notably, the bottom layer 102B is a side that may be placed against a part of the body of the subject for the biological sample, occupying the opposite top layer 102A on the implantable bioelectronic device 100, to interact with the damaged nerves/tissue of the subject and restore interrupted biological function. Notably, the top layer 102A and the bottom layer 102B are fabricated from for example a parylene derivative and gold, while the bottom layer 102B lacks the biological sample and the biodegradable hydrogel that are only present on the top layer 102A. As shown, the implantable bioelectronic device 100 consists of two layers (102A and 102B) of the base material 102 containing Au (Gold) tracks (namely, the electrode 104) and PEDOT:PSS microelectrodes. Au (Gold) tracks lead to connector pads to which a connecting flexible cable can be bonded. The two layers (102A and 102B) of the base material 102 contain at least one hole 106 to permit fluid flow and cell migration across the implantable bioelectronic device 100.

FIGS. 1B and 1C are enlarged views of the implantable bioelectronic device 100 of FIG. 1A. Here brightfield images show the biological sample 108 (human iPSC-derived myocytes) at Day 8 of culture as viewed with scale bars representing 465 μm (FIG. 1B) and 230 μm (FIG. 1C), respectively.

FIG. 1D is an illustration of the preparation of the implantable bioelectronic device 100 of FIG. 1A for cell culture. As shown, the hydrogel 110 coats the cultured biological sample 108 (human iPSC-derived myocytes). Upon cell culture, the implantable bioelectronic device 100 is removed from the cell culture plate 112 comprising polydimethylsiloxane (PDMS) wells 112A, and implanted into a subject at a desired location (such as a rat peripheral nerve).

FIG. 1E is a graphical representation of the electrode impedance for a control device and the implanted implantable bioelectronic device 100 of FIG. 1A at different weeks of implantation. As shown, over 4 week period post-implantation, the electrode impedance increases for the functional electrodes 104 of the implantable bioelectronic device 100 comprising biological sample cultured thereon at 1000 Hz every week over a 4 week period post-implantation. As shown, the electrode impedance is highest at week 4. A control device that lacks the biological sample 108 cultured thereon shows no impedance.

FIG. 1F is an illustration of an experimental timeline showing the transfer of the implantable bioelectronic device 100 of FIG. 1A from an in vitro step to an in vivo step. As shown, the biological samples 108 are seeded onto the implantable bioelectronic device 100 at day zero. Herein, the biological sample 108 is human iPSC. After 48 hrs (Day 2) the differentiation process is initiated and the human iPSC differentiate into muscle cells (myocytes). At Day 8 the muscle cells mature into myotubes, therefore between Day 8-10 is the optimal timing for the implantation of the implantable bioelectronic device 100 into a subject, such as a peripheral nerve rat model. The implantable bioelectronic device 100 is then implanted for a period of 4 weeks. During this 4 week period, live chronic and acute electrophysiology recordings are performed on the subject. As shown, during implantation, the cell-laden side of the implantable bioelectronic device 100 is transferred a few centimetres towards the midline of the subject and anchored subcutaneously to support cell survival for at least 7 days after implantation and to further be receptive to axon growth and innervation.

It will be appreciated that the term “implantable bioelectronic device” refers to a biohybrid device that comprises cells, such as human-derived cells, for example myocytes, combined with (such as by way of seeding thereon) an implantable electronic device, and the term “control device” refers to an implantable electronic device lacking cells, i.e. no human-derived cells, such as myocytes, are seeded on to the control device.

It will be appreciated that hydrogels are soft, water-swollen materials that may mimic the mechanical and chemical properties of biological tissues. They may be beneficially used in implantable bioelectronic devices of the present disclosure, for example in flexible implantable bioelectronic devices, as encapsulating materials for sensors and electronic components, as well as for drug delivery and tissue engineering applications. Hydrogels may also be designed to respond to external stimuli such as temperature, pH, and light.

Biodegradable hydrogel materials that may be used in implantable bioelectronic devices of the present disclosure, for example in flexible implantable bioelectronic devices, for example for implementing the biodegradable hydrogel 110, 1108, optionally include one or more of:
(i) Poly(ethylene glycol) (PEG): PEG hydrogels provide good biocompatibility, low toxicity, and may be tuned to exhibit various mechanical properties depending on a degree of crosslinking within the PEG hydrogels;
(ii) Poly(acrylic acid) (PAA): PAA hydrogels may swell and shrink in response to changes in pH, making them useful for pH-sensitive drug delivery and biosensing applications;
(iii) Alginate: Alginate hydrogels are derived from seaweed and exhibit good biocompatibility, low toxicity, and may be used for cell encapsulation and tissue engineering applications;
(iv) Chitosan: Chitosan hydrogels are derived from chitin, which is a natural polymer found in crustacean shells. Chitosan hydrogels exhibit good biocompatibility, antibacterial properties, and may be used for wound healing and drug delivery applications; and
(v) Gelatin-based hydrogels: Gelatin-based hydrogels are derived from collagen and exhibit good biocompatibility and mechanical properties. The gelatin-based hydrogels may be used for cell encapsulation, tissue engineering, and drug delivery applications.

Overall, aforesaid biodegradable hydrogels may exhibit unique properties and advantages for implantable bioelectronic devices, for example in flexible implantable bioelectronic devices; such unique properties include biocompatibility, responsiveness to external stimuli, and a characteristic in use to mimic biological tissues. Moreover, the hydrogels may be used alone or in combination with other materials to create multifunctional and highly adaptable implantable bioelectronic devices.

These biodegradable hydrogel materials may be used in various combinations and configurations to manufacture implantable bioelectronic devices, for example in implantable bioelectronic devices, for a range of applications; such a range of applications include wearables, implantable medical devices, and soft robotics. A specific choice of one or more materials that are used will depend on the requirements of the implantable bioelectronic device, such as at least one of: an intended use of the implantable bioelectronic device, performance requirements of the implantable bioelectronic device, and biocompatibility considerations that pertain to the implantable bioelectronic device.

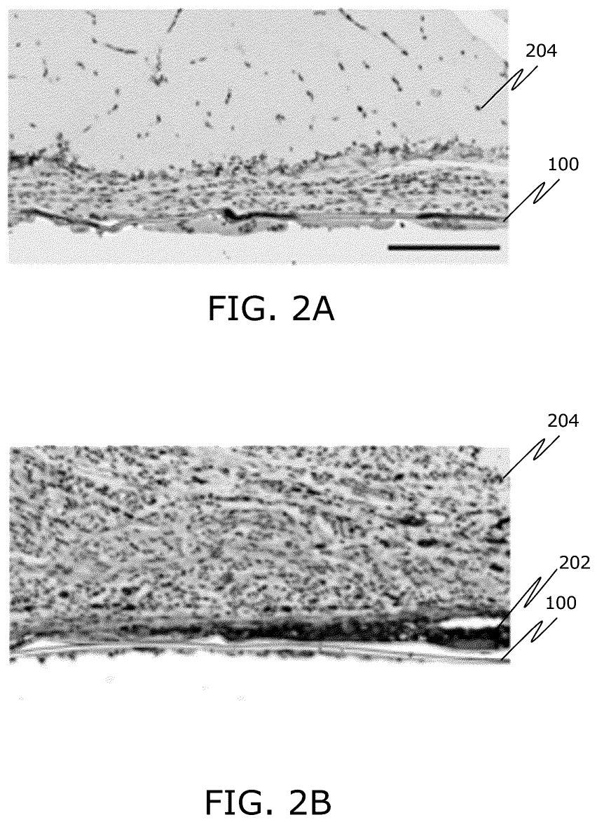

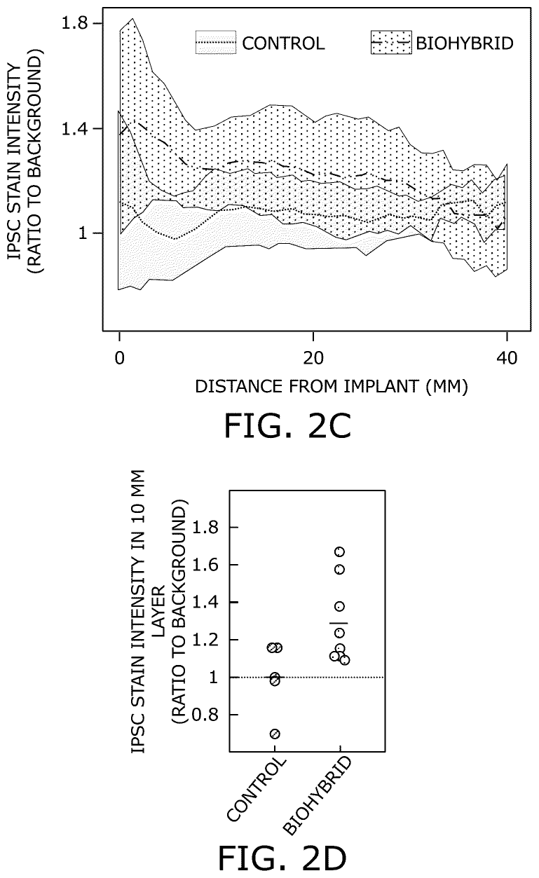

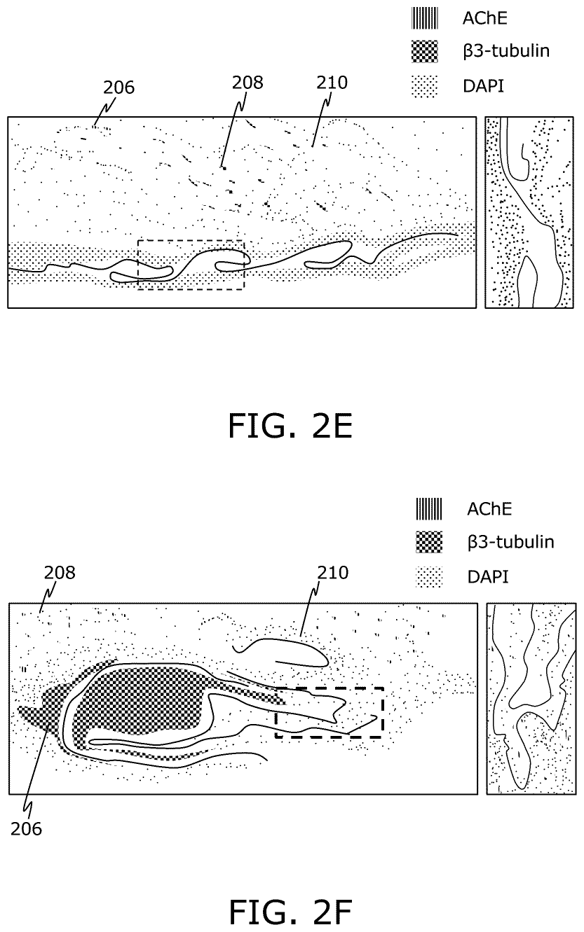

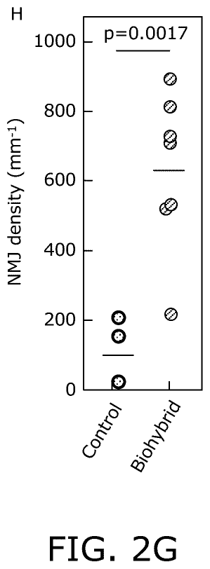

FIGS. 2A and 2B are illustrations of the implantation of a control device and the implantable bioelectronic device 100 of FIG. 1A, respectively, in accordance with an embodiment of the invention. As shown in FIG. 2B, 28-day post-implantation, the implantable bioelectronic device 100 shows the survival of human iPSC-derived muscle cells 202 and the ability to restore the lost biological function in the subject. However, the control device shows no restoration of lost biological function due to the lack of biological sample thereon. Grey traces depict human iPSC-derived muscle cells 202 and black traces depict host cells 204. Notably, high cell density tissue in proximity to the biohybrid image is part of the regenerated forearm nerve bundle in the subject.

FIGS. 2C and 2D are graphical representations of the biological sample stain intensity with respect to the distance thereof from the implantation site of the control device and the implantable bioelectronic device 100 of FIG. 1A, respectively. As shown in FIG. 2C, the human nucleoli stain intensity (ratio to background) for the implantable bioelectronic device 100 (dashed line) decreases over distance from the implant while the human nucleoli stain intensity (ratio to background) for the control device (dotted line) is nearly constant over distance from the implant.

FIG. 2D shows the average human nucleoli stain intensity in the 10 μm layer closest to the implant. As shown, the implantable bioelectronic device 100 shows a significantly enriched presence of human iPSCs compared to the control device (statistical values were calculated via a Student's t-test). The circles in the plot represent the average in individual subjects (rats), the bar indicates the mean of the group.

FIGS. 2E and 2F show images of immunofluorescence stains of the implantable bioelectronic device 100 and the control device (lacking cells) for axons (β3-tubulin) 206 and neuromuscular junctions (NMJs) (AChE-acetylcholinesterase) 208, as well as cell nuclei (DAPI) 210 28 days post-implantation. The host nerve is found in close proximity to the implantable bioelectronic device 100 but not the control device. An abundance of NMJs 208 are seen in immediate proximity to the implantable bioelectronic device 100 but not the control device. The right panels of the FIGS. 2E and 2F show images shown inset of the portions under dashed lines. The white arrowheads in the inset indicate examples of NMJs 208. As shown, the position of the implant is marked by a white line in the images. Scale bars: 500 μm (left panel) and 100 μm (insets, i.e. right panel).

FIG. 2G shows quantification of neuromuscular junctions (NMJ) 208 density in a subject. Herein, the circles represent an average in individual rats, the black bar indicates a mean of the group. P values are calculated via Student's t-test.

Notably, the FIGS. 2E-G confirm the presence of the neuromuscular junction (NMJ) 208 formation at the interface of the implantable bioelectronic device 100 with host peripheral nerve tissue, but not of the control device lacking myocytes, and suggest the innervation of the implantable bioelectronic device 100 by host motor axons. Moreover, although no major differences in host tissue appearance and cell density may be seen across the implantable bioelectronic device 100 and the control device, the nerve to which the implantable bioelectronic device 100 is attached may often be found in very close proximity to the implantable bioelectronic device 100, but not the control device.

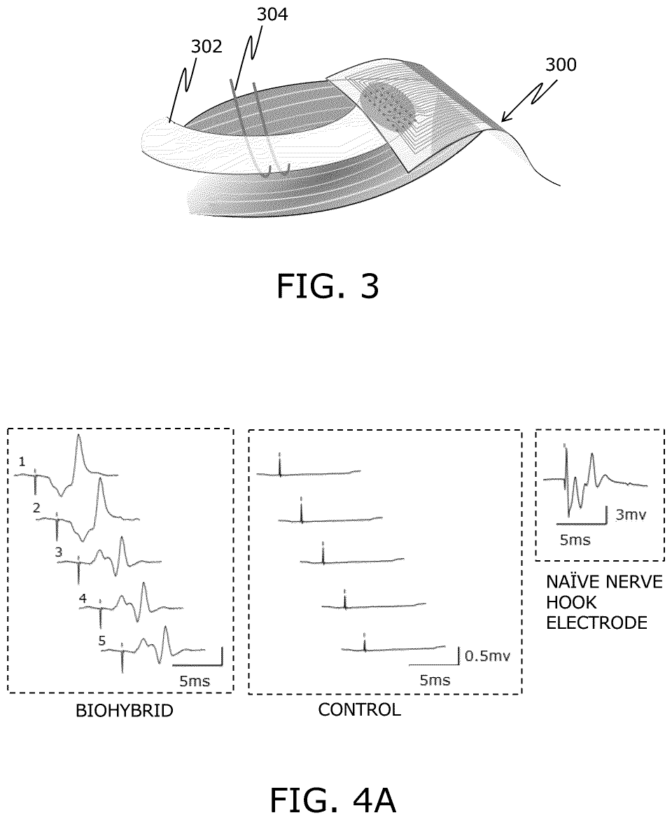

FIG. 3 is a schematic illustration of an implantation of an implantable bioelectronic device 300, in accordance with an embodiment of the invention. The implantable bioelectronic device 300 enables transected host nerves 302 to retain healthy nerve electrophysiology 28 days post-implantation. As shown, a transected host nerve 302 (here, forearm nerve bundles) chronically implants either the implantable bioelectronic device 300 (such as the implantable bioelectronic device 100 of FIG. 1A) or a control (lacking iPSCs) implantable bioelectronic device. The nerve response to electrical stimulation is evaluated using the chronically-implanted devices, i.e. either the implantable bioelectronic device 300 (such as the implantable bioelectronic device 100 of FIG. 1A) or a control (lacking iPSCs) implantable bioelectronic device. In this regard, a nerve response to electrical stimulation is evaluated using a pair of hook electrodes 304 around the forearm nerve bundle approximately four centimetres above the point of transection and implantation.

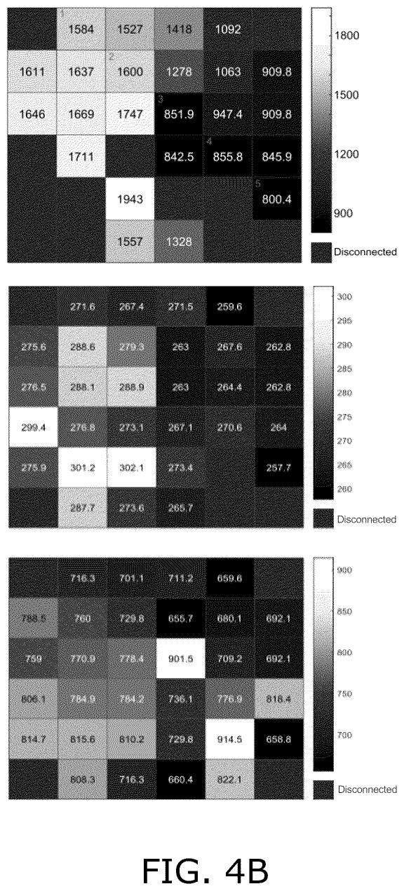

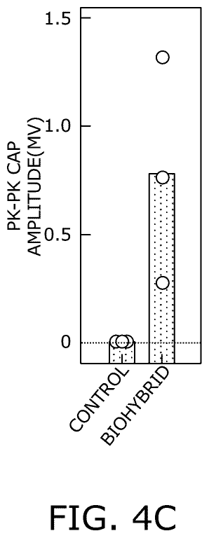

FIG. 4A represents the response of a stimulated nerve of the implantable bioelectronic device (left panel) in comparison to a control device (right panel). As shown, compound action potentials (CAPs) are recorded from the implantable bioelectronic device but not from the control device. As shown, traces of nerve response to 100 μA, 0.1 ms stimulation pulses, are taken from different electrodes of the implantable bioelectronic device. CAP recorded from a naïve nerve hook electrode are for comparison, wherein the naïve nerves are non-transected. Notably, the naïve nerve hook electrode mimics signals from a nerve in a conscious rat, as signals are not fired when the rat is under anaesthesia. CAPs recorded by electrodes within the implantable bioelectronic device each likely from a segment of the nerve cross-section are similar to those recorded from a whole intact nerve. The stimulation time is identified by squares in the traces of each of the nerve responses.

FIG. 4B is a representation of an average peak-to-peak compound action potential (CAP) amplitude recorded by the implantable bioelectronic device of FIG. 4A. Notably, higher CAP amplitudes (voltages) are represented by lighter colours in the heatmap, with exact values represented by the numbers. Disconnected sections have an electrode impedance>500 kΩ. The traces from the implantable bioelectronic device of FIG. 4A taken are indicated by left-corner numbers 1 through 5 (top panel). Non-uniformness of CAP recordings with higher amplitudes loosely matching dimensions of the nerve is suggestive of selective recordings of the nerve from the implantable bioelectronic device.

FIG. 4C is a graphical representation of peak-to-peak compound action potential (CAP) amplitude recorded by the 28-day implanted implantable bioelectronic device and the control device of FIG. 4A. The implantable bioelectronic device and the control device are stimulated with a 100 μA pulse stimulation. As shown, the control device exhibits no CAPs in response to stimulation, while the implantable bioelectronic device exhibits CAPs in response to stimulation. The circles represent values for each subject (mean across the entire MEA), the bar indicates the mean of the whole group.

Notably, the amplitude of the CAPs recorded by the implantable bioelectronic device electrodes is lower than seen in hook electrodes around intact nerves, likely as a consequence of the smaller size of the former and indicative that each of these recordings are from only a portion of the nerve.

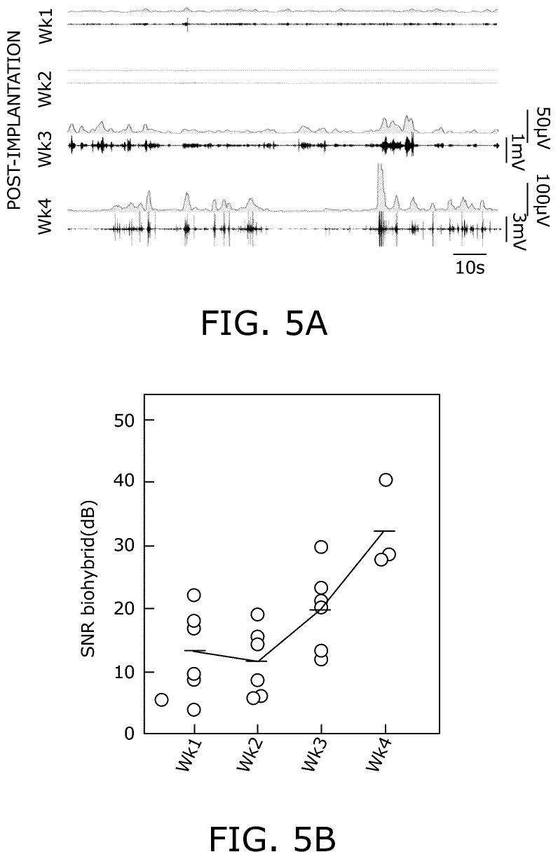

FIGS. 5A and 5B are representations of the nerve signal traces recorded by the implantable bioelectronic device of FIG. 4A over 4 weeks post-implantation and a signal-to-noise ratio (SNR) of the recorded traces, respectively. As shown in FIG. 5A, the nerve electrical recordings from the implantable bioelectronic device progressively improves over 4 weeks post-implantation, coinciding with nerve regeneration and synaptic integration with the implanted human iPSC-derived muscle cells (biological sample). Herein, black traces show bandpass filtered (0.2-4 kHz) time traces recorded from a pair of electrodes in the MEA (bipolar configuration) and grey traces show root-mean square (RMS) of black traces with 0.5 s rolling window. As shown, the nerve signal amplitude increases over implantation period.

As shown in FIG. 5B, the circles show mean value per bipolar electrode and the line shows mean of group, N=2 subjects (rats). The statistical comparison is done via a one-way ANOVA followed by Tukey post-hoc. The P values not shown are >0.05. Within the first two weeks of implantation, a little activity is observed in the awake animals. By the third and in particular the fourth weeks, however, signals greatly and significantly increase in amplitude such that at weeks 1 to 4:12.9, 11.3, 19.7, 32.0 dB mean signal-to-noise ratio, respectively. It will be appreciated that the readings were recorded in real-time in freely moving animals. Moreover, recordings are taken from the same electrodes throughout the experimental timeline.

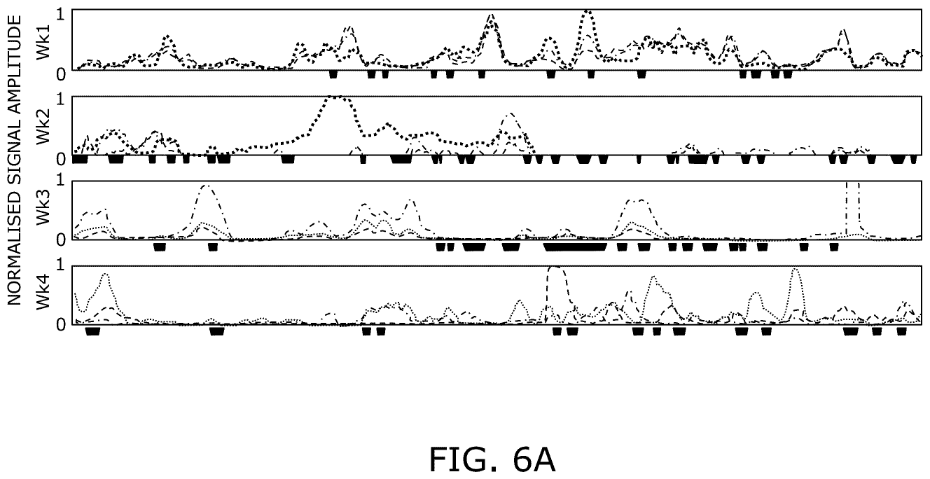

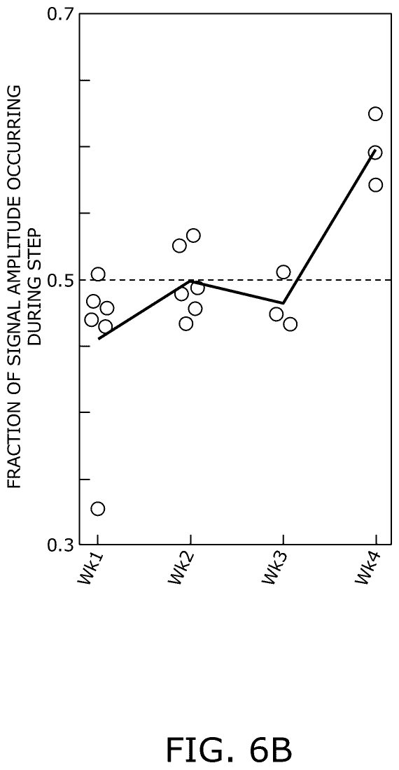

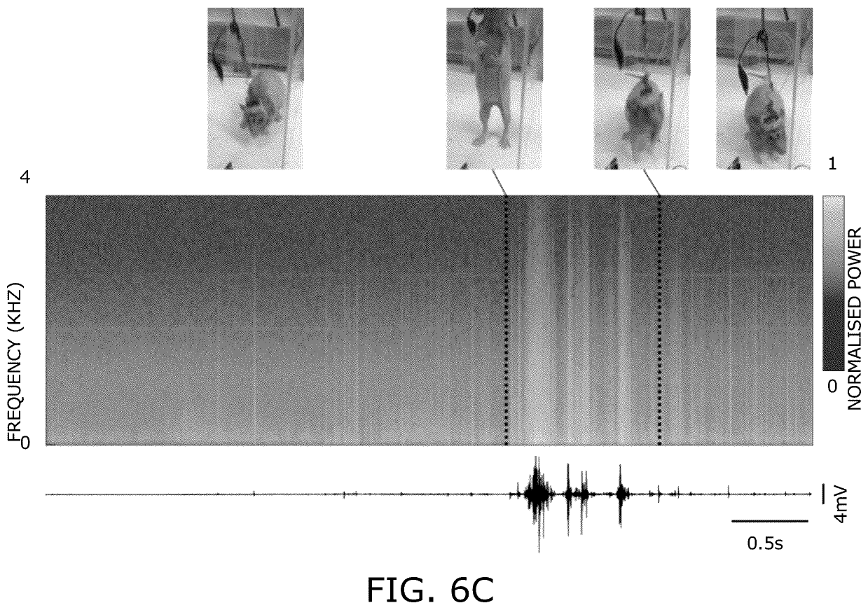

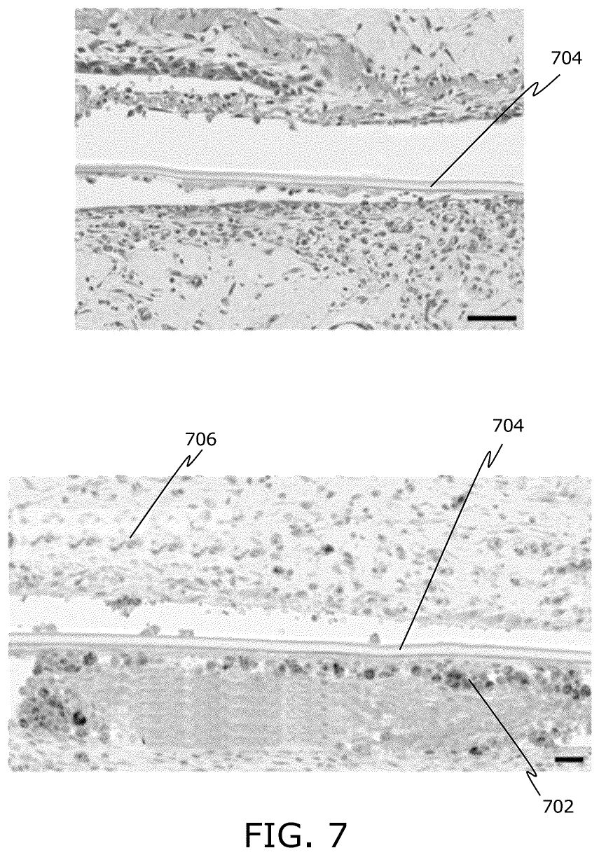

FIG. 6A is a graphical representation of root mean square (RMS) time traces from 3 bipolar electrodes recorded by the implantable bioelectronic device of FIG. 4A over 4 weeks post-implantation. As shown, the 3 bipolar electrodes (dashed line, dotted line and dashed-dotted line) are normalised to range from 0 to 1 for each week. Points when the subject (the rat) is reaching out and stepping with the implanted body part (paw) are indicated by black squares under each trace. Increased correlation between the recorded activity and the body part movement, indicative of good nerve recording from said body part, develops at week 4 post-implantation, with less activity seen outside of stepping events.

FIG. 6B is a graphical representation of quantification of correlation between the recorded RMS time traces of FIG. 6A and stepping events. The circles show the mean fraction of RMS activity occurring during steps. These values are adjusted to account for the time stepping/not stepping in that session. The line shows the mean of the group, N=2 subjects (rats). Statistical comparison is done via a one-way ANOVA followed by Tukey post-hoc. The P values not shown are >0.05.

FIG. 6C is a representation of the time-frequency spectrogram and time trace of a recording from a bipolar electrode of FIG. 6A at week 4 post-implantation. As shown, high activity is seen when the subject steps up and leans with paws (splaying of digits visible in non-implanted forearm, which retains an intact forearm nerve bundle, delineated by black dotted lines) on chamber walls. Comparatively less activity is seen before and after this event, as the animal remains on the bottom of the chamber, indicating that recorded activity is driven by attempted paw use. Notably, in a freely moving chronic biohybrid rat group, at early implantation timepoints recorded activity appeared to be independent of movement of the operated limb. However, by week 4, post-implantation activity increasingly correlated with the movement of the implanted paw.

FIG. 7 is an illustration of a biological sample 702 surviving 7 days post-implantation of the implantable bioelectronic device 704. As shown, the biological samples 702 stained dark grey (using a human nucleoli dye) confirms survival post-implantation along with the host cells 706 that are in light grey.

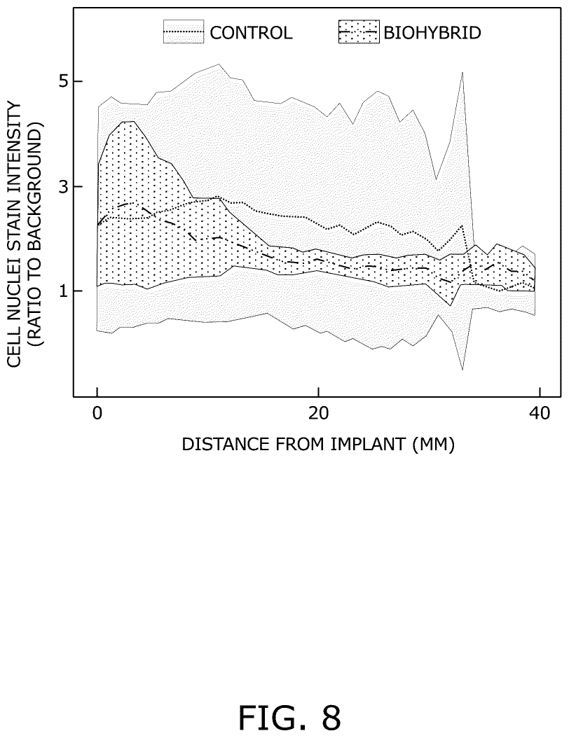

FIG. 8 is a graphical representation of a cell nuclei stain intensity over a distance from 28 day-implanted implantable bioelectronic device 100 of FIG. 1A and a control device. As shown, all bioelectronic device implants show an increased cellular density close to the implant surface. Notably, no major differences in host cell density are seen across the implantable bioelectronic device 100 of FIG. 1A and the control device.

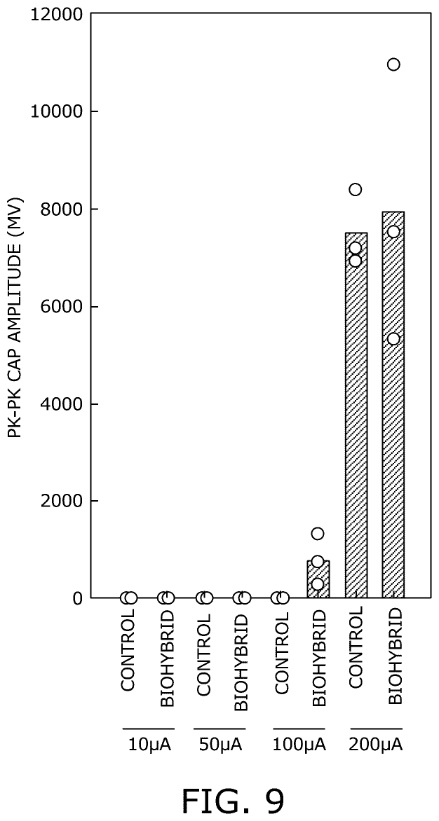

FIG. 9 is a graphical representation of peak-to-peak compound action potential (CAP) amplitude recorded by the 28-day implanted implantable bioelectronic device 100 of FIG. 1A and a control device for 0.1 ms pulses of a range of amplitudes. The circles represent values for each subject (mean across the entire MEA), and the bar indicates the mean of the whole group. While all nerves implanted with the implantable bioelectronic device 100 produced CAPs in response to 100 μA stimulation pulses, their average amplitudes differed, suggesting a degree of variability in implantable bioelectronic device 100 integration across animals. Consistent with normal nerve behaviour, stimulation with pulses of a lower amplitude than activation threshold resulted in no CAP. Stimulation with higher amplitude pulses eventually resulted in CAPs recorded in both groups, the implanted implantable bioelectronic device 100 and the control device. Notably, CAPs recorded by the control device under these stimulation conditions consisted exclusively of an H-reflex, and may have instead been mediated by EMG from reflex activity of other nerves at the same cervical level as the forearm nerve bundle.

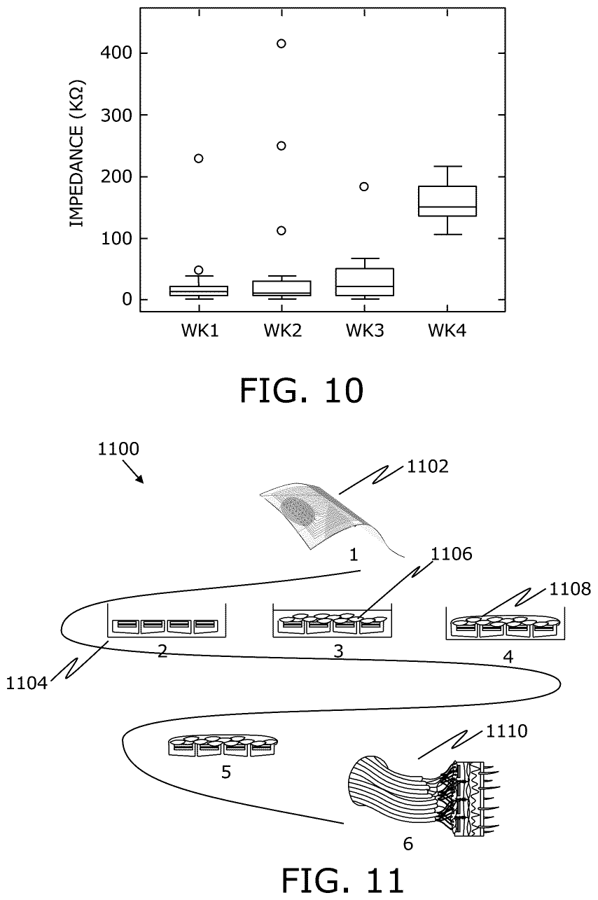

FIG. 10 is graphical representation of quantification of impedance of the implanted implantable bioelectronic device of FIG. 1A over 4 weeks of implantation. As shown, impedance increases for the functional every week over a 4 week period post-implantation with the impedance being highest at week 4.

FIG. 11 is a schematic illustration of a step-by-step process 1100 involved in culturing a biological sample onto an implantable bioelectronic device 1102, in accordance with an embodiment of the invention. As shown, firstly, the implantable bioelectronic devices 1102 are fabricated using photolithography techniques, and are then temporarily adhered to cell culture plates 1104 for the biological sample, such as for example the human iPSC-derived muscle 1106 (as used in this example), culture thereon. Once the human iPSC derived muscle 1106 are mature a biodegradable hydrogel, such as a fibrin hydrogel 1108 (as used in this example), is polymerised on top of the implantable bioelectronic device 1102 and mature human iPSC-derived muscle 1106 to ensure they are not damaged during surgical implantation. The device is then removed from the cell culture plate 1104 and implanted into the rat peripheral nerve 1110.

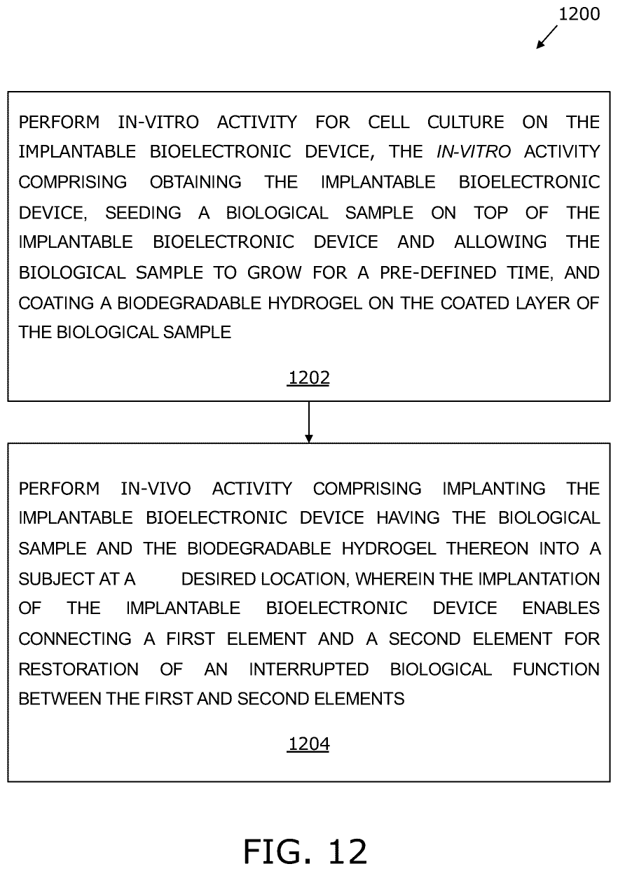

Referring to FIG. 12, illustrated is a flowchart 1200 of the steps of a method of using an implantable bioelectronic device, in accordance with an embodiment of the invention. At a step 1202, an in vitro activity is performed for cell culture on the implantable bioelectronic device. The in vitro activity comprises obtaining the implantable bioelectronic device, seeding a biological sample on top of the implantable bioelectronic device and allowing the biological sample to grow for a pre-defined time. At step 1204, an in vivo activity is performed. The in vivo activity comprises implanting the implantable bioelectronic device with the biological sample and the hydrogel thereon into a subject at a desired location, wherein the implantation of the implantable bioelectronic device enables connecting a first element and a second element for restoration of an interrupted biological function between the first and second elements.

The steps 1202 and 1204 are only illustrative and other alternatives can also be provided where one or more steps are added, one or more steps are removed, or one or more steps are provided in a different sequence without departing from the scope of the claims herein.

It will be appreciated that the new category of neural interface, which combines flexible electronics and human iPSC-derived cells, can survive in a rat model for up to 28 days and integrate with the host tissue forming neuromuscular junctions (NMJs) and can be used to restore and drive functionality through the implantable bioelectronic device 100. The neural interfaces of such implantable bioelectronic devices show superior electrophysiology recording properties and tissue integration compared to standard neural interfaces (flexible electronics without cells). This novel strategy enables axon fibre type-specific recording selectivity as well as potentially significant increases in spatial resolution and opens the door for the development of the next generation of smart prosthetics for severe peripheral nerve injuries that often lead to amputations and painful neuroma development. Beneficially, the implantable bioelectronic device 100 offers unique advantages in the context of amputation treatments by providing higher signal quality through the biological amplification step performed by the innervated myocytes. Moreover, the selection of the transplanted cell type offers a unique mechanism to interact with a specific type of axon—in the case of the implantable bioelectronic device 100, recording from motor axons specifically. The independent communication with axons transmitting different types of information may enable more flexible control and sensation in prosthetic systems, greatly improving their application in amputations. While conventional biohybrid devices conceptually rely on regeneration to integrate with host tissue, the implantable bioelectronic device 100 employs (i.e. uses) a minimalist two-dimensional design fabricated from ultra-flexible materials and contained a cell biohybrid layer-two factors associated with greatly decreased FBR. Moreover, as the regenerative design implemented did not require axons to regenerate through the implant body itself, the implantable bioelectronic device 100 may avoid the long-term stability issue encountered by other conventional regenerative designs.

Moreover, the timeline of the appearance of high signal amplitude recordings in the implantable bioelectronic device 100 provides an informative outline of the tissue integration events occurring around these devices in vivo. The first week following nerve transection and device implantation yields low quality signals as damaged axons retract and begin regenerating. The presence of a small, millimetre-size gap between the nerve stump and the device created during implantation will lead to the growth of a nerve scaffold to serve as a bridge before axon regeneration crossing takes place (weeks 1-2). The arrival of axons to the proximity of electrodes may produce an increase in signal amplitude (week 3). However, NMJ formation with biohybrid myocytes will have to occur before myocytes summate their electromyogram activity to that of axons to improve the signal amplitude, a process which may last over a week (week 3-4). The biohybrid signal recording evolution and integration of nerve and implant at 4 weeks post-implantation (as shown in FIGS. 2E-G) supports this timeline of events.

Moreover, looking ahead at the wider impact of this technology, the biohybrid neural interfaces could (may) be adapted through the use of different transplanted cell types such as those with neuronal or glial phenotypes to promote integration with other types of tissues such as the brain and spinal cord. This could (may) potentially extend the scope of treatments addressable by this technology to conditions such as stroke, traumatic brain injury, and spinal cord injury. By selecting the appropriate implant design and cell type, customisable biohybrid neural interfaces could (may) be generated to meet patients' individual requirements. Furthermore, the combination of an implanted device and cells allows, through bespoke genetic modifications of parental iPSC for instance, for the use of local drug delivery, for immunosuppression or growth factor delivery.

### EXAMPLE

In an exemplary implementation of the disclosed method, the biological sample is seeded onto the flexible base material, arranged in a cell culture plate, such as a PDMS cell culture plate, at day zero. After 48 hrs (i.e. at day 2) the differentiation process of the biological sample initiates. At day 8 myotubes mature and a biodegradable hydrogel is polymerised on top of the grown cells and the implantable bioelectronic device to ensure they are not damaged during surgical implantation. The implantable bioelectronic device is then removed from the cell culture plate and implanted at a desired location in a subject, such as a peripheral nerve in a sutured forearm of rats between day 8-10. The implantable bioelectronic devices are then left implanted for a period of 4 weeks. During this 4-week period, live chronic and acute electrophysiology recordings are performed on the subject. During the acute electrophysiology, i.e. 28 days post implantation, the implanted nerve is electrically stimulated with a 100 μA pulse and a compound action potential (CAP) is recorded from the implantable bioelectronic device, but not from control implants that lack the implantable bioelectronic device. Notably, the CAP features recorded are consistent with those found in intact sensorimotor nerves, indicating healthy nerve physiology in the transected forearm nerves bundle (comprising combined ulnar and median nerves) implanted with the implantable bioelectronic device.

In an alternative implementation, the cell culture process comprises seeding iPSC clusters on the biodegradable hydrogel layer previously laid down on the MEA surface, followed by induction of differentiation 48 hours later. This resulted in the formation of mature myotubes on the surface of the biohybrid device by Day 8. The iPSC-derived myotubes exhibited acetylcholine-induced contraction at Day 8 of culture. Notably, prior to cell seeding, there is reported a 97% yield, 1.84±2.20 kΩ and post Week 4 in vivo, there is reported a 25% yield, 159.00±35.80 kΩ, mean±SD).

Device Fabrication: The iPSCs are cultured on thin, flexible parylene-based multi-electrode arrays (MEAS). Herein, a 2 μm-thick parylene C layer is deposited on silicon wafers (100 mm outer diameter, thickness of 1 mm) using an SCS Labcoter 2. The MEAs are fabricated using standard photolithography techniques to contain 32 conducting polymer (PEDOT:PSS) electrodes arranged in a symmetrical grid, such as a 2 mm×2 mm area within the larger parylene-based flexible base material. The flexible base material also comprises a plurality of circular holes (openings) to permit the growth of vasculature from the back of the parylene-based flexible base material of the implantable bioelectronic device and support cell survival post-implantation of the implantable bioelectronic device.

Gold (Au) electrodes and interconnects are patterned through a metal lift-off process. AZnLOF9260 photoresist is spin-coated at 3,500 r.p.m. on the substrate, baked at 110° C. for 120s, exposed to ultraviolet light using a Karl Suss Contact Mask Aligner MA/BA6 and developed with AZ 760 MIF developer. A 10-nm-thick Ti adhesion layer, followed by a 100-nm-thick Au (Gold) layer, is deposited (Angstrom EvoVac Multi-Process) and patterned by soaking the substrate in a bath of acetone for 10 minutes. A second 2 μm-thick layer of parylene C (insulation layer) is deposited, followed by spin coating a layer of soap solution (2% Micro-90 diluted in deionized water) before an additional sacrificial 2 μm-thick layer of parylene C (for the subsequent peel-off process) is also deposited. The layers of parylene are then patterned with another layer of positive photoresist (AZ9260) to shape the PEDOT:PSS electrodes and contact pads. This photoresist is then dry etched using reactive ion etching to expose electrodes and contact pads. Once etched, a thin film of PEDOT:PSS is spin-coated onto electrodes (as previously described by Rivnay et al. 2016). The solution is spin-coated 3 times with soft bakes in between for 60s at 120° C. After the final spin coat there is a hard bake for 1 hr at 130° C. Post baking the wafer is left overnight in DI (deionized) water to remove excess PSS. The following day the sacrificial layer of parylene C can be removed, leaving the finished device ready for use. Devices at this stage could (may) either be implanted as control devices, or taken through a cell culture protocol to produce a layer of myocytes on fibrin hydrogel before their use in vivo (the implantable bioelectronic device).

Biological Sample: In this regard, OPTI-OX human induced pluripotent stem cells (iPSCs) is selected as the biological sample for the biohybrid cell population. Notably, these cells differentiate into myotubes from Day 8 in cell culture post doxycycline and retinoic acid induction and regenerate within 3 weeks after injury. At this time point, the cultured myotubes are generally considered fully differentiated and receptive to axon growth and innervation. Moreover, said cells are well-suited to host sensorimotor nerves, whose motor axons typically innervate muscle tissue, while their human iPSC-derived nature makes their use clinically translatable. It will be appreciated that the biological sample may also differentiate into other cell types, including neurons. However, the disclosed method employs only pre-differentiated biological samples desired for a specific application.

Cell culture: Glass wells or custom-made polydimethylsiloxane (PDMS) wells are attached to the MEAs using PDMS as a glue. The devices are plasma treated at 25 W for 1 min to make the surface hydrophilic for cell culture. The inside of the well is kept wet from this point on with DI (deionized) water. The implantable bioelectronic device is entirely sterilized for a minimum of 30 min in 70% ethanol and rinsed with Dulbecco's phosphate-buffered saline (DPBS).

OPTi-MyOD hiPSCs are defrosted and expanded in Essential 8™ Flex Medium for approximately 3 to 4 days in 6 wells plates. This gave approximately 1.5 million cells/mL. OPTi-MyOD hiPSCs are seeded onto devices with densities of 100,000 cell/cm. Differentiation is initiated 48 hours after cell seeding.

The MyoD media is supplemented with fresh 1 μg/mL doxycycline (Sigma) and 1 μM Retinoic acid (Sigma) and 40 ng/ml of FGF2 (R&D). The cell culture media is changed every day from day 0 to day 5. From day 6 onwards, MyoD media is supplemented only with 1 μM Retinoic acid, 3 μM of CHIR99021 (Tocris) and 10% KOSR (ThermoFisher) and no doxycycline.

Biodegradable hydrogel preparation: A fibrinogen stock solution is prepared at a concentration of 37.5 mg/ml in HEPES-buffered saline (HBS: 20 mM HEPES, 150 mM NaCl, pH=7.4) by slowly dissolving fibrinogen (F8630, Sigma Aldrich) for 2 hours at 37° C. (solution named SOL-FG) to result in Fibrinogen Solution (SOL-FG). Filter SOL-FG with a 0.22 μm filter for sterilisation (and removal of any aggregates). Any further dilutions are performed in sterile HBS. Next, a Calcium Thrombin Solution (SOL-CaTh) is created containing 3 U/mL thrombin and 60 mM calcium ions. A 120 mM stock solution of CaCI₂ in HBS is prepared. A thrombin (T9549, Sigma Aldrich) stock solution of 6 U/ml in HBS is prepared and kept on ice. A thrombin and CaCl₂) solution is prepared by mixing equal volumes of SOL-Th and SOL-Ca, obtaining a solution SOL-CaTh and kept on ice. A solution containing 1,000,000 cells/mL in cell culture media (SOL-Cells) is prepared and is used to coat cells that had been grown and differentiated on ParC based implantable bioelectronic devices. For the production of 150 μL gels (scale accordingly) 54 μL of SOL-FG and 54 μL of HBS are mixed, and 54 μL of SOL-CaTh is added to the fibrinogen-cell mix and immediately 150 μL of the gelling solution is pipetted into the desired vessel and incubated at 37° C. for 2 hours to allow gelation to occur. Once cells and fibrinogen are mixed, these solutions are used within 15 mins as cells/residual thrombin in cell culture media will start gelling the solutions. Final concentrations: FG=3.125, 6.25, 12.5, 25 mg/mL, Th=1 U/mL, CaCl₂)=20 mM.

Animal procedures: It will be appreciated that all animal work for developing embodiments of the present disclosure was performed in accordance with the prescribed procedures, such as the UK Animals (Scientific Procedures) Act 1986. All work was approved by the United Kingdom Home Office (project licence number PFF2068BC), and the Animal Welfare and Ethical Review Body of the University of Cambridge. 190-240 g Hsd: RH-Foxn1rnu athymic nude rats (Envigo, France) was used in this study. Surgical procedures were performed under isoflurane anaesthesia, with the animal's body temperature regulated using a thermal blanket.

For implantation of the implantable bioelectronic device, an incision is performed with a sterile knife (or blade), at the desired location in the subject, i.e. at elbow height in the forearm nerve bundle (combined ulnar and median nerves) under the triceps muscle in rats, immediately prior to device implantation to promote tissue regrowth and angiogenesis in the vicinity of the implant. The scoring is done and then the proximal nerve stump is sutured, using a 9-0 nylon suture (Ethicon), the cell-laden side of the implantable bioelectronic device that is transferred a few centimetres towards the midline of the animal and anchored subcutaneously above the latissimus dorsi muscle. Beneficially, said implantation strategy can support cell survival for at least seven days after implantation. Animals are allowed to recover from the surgical procedure and provided with analgesics (Meloxicam, Carprofen) for two days post-implantation, as well as immediately prior to surgery. Animals are housed in groups of three or four with ad libitum access to food and water. Control implants consisting of the same flexible implantable bioelectronic device, but lacking myocytes are implanted in an identical way. The implantable bioelectronic devices are kept sterile throughout the cell culture period, and are therefore not sterilised again prior to implantation. Control devices are sterilised in 70% ethanol for 30 min (minutes) and rinsed with sterile saline (solution).

Device electrical characterisation: Impedance measurements are completed with a potentiostat (Autolab PGSTAT128N) in a three-electrode configuration. An Ag/AgCI electrode (Ag=Silver; Au=Gold; CI=Chlorine) is used as the reference electrode, a Pt (Platinum) electrode is the counter electrode and the working electrode is the recording electrode of the MEA. The characterization is performed in a DPBS solution.

Electrophysiology recordings under anaesthesia: 28 days post-implantation, the implantable bioelectronic device is stimulated using an acquisition and stimulation system (a 32-channel RHS headstage and RHS Stim/Recording Controller, Intan Technologies, USA) in vivo using a pair of hook electrodes (platinum Pt wire hooks) around the forearm nerve bundle approximately 4 centimetres above the point of transection and implantation. The hooks are connected to the same acquisition and stimulation system. The nerve is stimulated using a 0.1 ms duration pulse, an activation threshold of 10, 50, 100 or 200 μA, and 20-30 stimulation pulses delivered for each amplitude. The MEA connections are externalised through a headcap.

Stimulation data is recorded (and amplified (X192) and digitised) using the 4 week implanted implantable bioelectronic device. To minimise EMG noise from nearby musculature, bipolar recording electrodes are set up between pairs of electrodes across the MEA. Notably, a compound action potential (CAP) is recorded from rats implanted with the implantable bioelectronic device, but not controls. Notably, the CAP consists of a peak with an approximately 2 ms delay (corresponding to a conduction speed of ˜20 m/s), consistent with Aa/B fibre activation, followed by a later peak, likely corresponding to reflex activity initiated by sensory fibre activation (H-reflex). These CAP features are consistent with those found in intact sensorimotor nerves, indicating healthy nerve physiology in the transected forearm nerves implanted with the implantable bioelectronic device. Notably, H-flex (or Hoffmann's reflex) is an electrical stimulation-based reflectory reaction of sensory fibres. Typically, H-reflex test is indicative of muscle response to electrical stimulation thereof by an electrical stimulator.

Notably, the recordings are measured from animals in both an anesthetized state and in an awakened state (where they freely roam around in a transparent area of 0.3 m×0.3 m) thereof. Analysis of the peak-peak amplitude of the response to stimulation in the raw recorded signals is carried out in Spike2 (Cambridge Electronic Design, UK, v9.04b) using a custom script. These referenced signals are then bandpass-filtered (0.5-4 kHz, 4^{th} order Butterworth filter). Signal-to-noise ratio (SNR) is calculated as the ratio of the variance during high signal relative to background activity, both identified manually, expressed as dB. RMS traces are produced from the referenced and bandpass-filtered signals by calculating the signals RMS (root-mean square) at 50 ms intervals and averaging the values using a 0.5 s rolling window. Normalised signal is calculated by normalising each RMS trace (single recording session) to range from 0 (background noise) to 1 (highest amplitude signal). The fraction of signal amplitude occurring during step is calculated by comparing the average normalised RMS value during stepping events, relative to the same average value outside of these events. All plotting and statistical tests are carried out in Matlab (Mathworks, R2021b).

In this regard, two experimental animal groups are selected on which the electrophysiology recordings are performed. In the first animal group, the animal is anaesthesised and terminal electrophysiology is performed. In the first animal group, a fake action potential (by using hook electrodes) is created to check whether the implantable bioelectronic devices after 4 weeks of implantation are capable of recording an action potential. In the second animal group, the animals are allowed to move freely. In such a case, no stimulation pulse is needed as the animal is awake, and the action potentials can be recorded in real time through the implantable bioelectronic device implanted for 28 days (4 weeks). It will be appreciated that the stimulation pulse is not always needed, it is needed only for the initial in vivo characterisation of the implantable bioelectronic device. Notably, for the purpose of both of these experiments, the implantable bioelectronic device acts as recording devices. However, in the future this technology could also be used to stimulate host nerve or brain tissue and/or record the same with or without stimulation.

Immunohistochemistry and Imaging: Tissue embedding and staining for implanted myocytes occurs on a Leica Bond RX autostainer. All steps are performed within a vacuum at 40° C. for 1 hour. The steps are as follows: a wash in 70% Ethanol, 90% Ethanol, four 100% Ethanol washes, three xylene washes, followed by 4 liquid paraffin wax steps at 63° C. The sections are first baked and de-waxed using Bond Dewax Solution (Leica Microsystems AR9222), then in the pre-treatment protocol Bond ER2 Solution at pH9 is used as the antigen retrieval solution (Leica AR9640) at room temperature. The Bond Wash used throughout is AR9590. For the staining protocol a Bond Polymer Refine Detection kit (Leica DS9800) is used. The kit includes the peroxidase block, post-primary, HRP polymer secondary antibodies, DAB and haematoxylin.

The staining protocol begins with 150 μL of peroxidase block added to the tissue samples and this is incubated for 5 minutes at room temperature. The sample is then washed with 150 μL of bond wash solution 3 times. Next, 150 μL of the primary antibody, mouse monoclonal to human nucleoli [NM95] (Abcam ab190710) is added for 60 minutes at room temperature. The sample is then washed with 150 μL of bond wash solution 3 times. 150 μL of the post-primary solution is incubated for 8 minutes at room temperature. Three 150 μL further bond washes are performed. Next, 150 μL of HRP polymer secondary antibodies is incubated on the sample for 8 minutes at room temperature. A 2-minute incubation with 150 μL bond wash solution is performed followed by a wash with 150 μL deionised water. Two washes with 150 μL DAB refine solution follow. 150 μL of Hematoxylin is next added and incubated on the sample for 5 mins. This is followed by washes with 150 μL deionized water, 150 μL bond wash solution and 150 μL deionized water. The samples are then ready to be imaged.

Imaging: Image analysis is performed in ImageJ software (National Institutes of Health). The edge of the tissue facing the device is traced by the user by hand and subsequently unfolded to become a flat image. Colour deconvolution is run to separate the implanted cells of interest (brown stain) from the host cells (blue) by the difference in histology stain colour. The stain intensity values are then imported into Matlab (Mathwords, R2021b) to produce a mean intensity over distance from the implant using a custom script. Following this, a 400×400 pixel box is chosen in the original image in a region of tissue far away from the device and the average background stain intensity is measured. The intensity profile is divided by this value to produce a normalised intensity for each stain.

Immunofluorescence (FIG. 2E-F) is carried out by performing an antigen retrieval step in a citrate buffer for 20 min (minutes). This is followed by blocking the samples in a phosphate-buffered saline solution of 5% donkey serum (D9663, Sigma) and 0.1% sodium azide at room temperature for 1 hour. Samples are then incubated with Anti-b3-tubulin (1/1000 dilution in blocking medium, ab7751, Abcam) and Anti-AChE (1/100 dilution, ab183591, Abcam). After 3 washes with a blocking medium, the samples are incubated with secondary antibodies (Donkey anti-Rabbit IgG (H+L) Highly Cross-Adsorbed Secondary Antibody, AlexaFluor 555; and anti-Mouse, AlexaFluor 488; Invitrogen) for 3 hours at room temperature, followed by Vector TrueVIEW Autofluorescence Quenching Kit (Vector Laboratories) for 3 minutes, mounted, and imaged in an Axioscan Slide Scanner (Zeiss). Images are then analysed in Image^{J} by performing a thresholding step over the AChE stain images and counting NMJs in an automated fashion using the Analyze Particles tool over an area of approximately 1 mm² in the immediate vicinity of the implanted device. Implanted devices are located through autofluorescence of the parylene-C in the DAPI channel.

All graph plotting and statistical comparisons are carried out in Matlab.

In the foregoing, optional examples of materials that may be used to make aforesaid implantable bioelectronic devices, for example to make flexible implantable bioelectronic devices, for example for implementing the flexible base material 102, include at least one of:
(i) Polyimide: Polyimide is a suitable material for use in manufacturing flexible bioelectronic devices due to its high thermal stability, excellent mechanical flexibility, and good chemical resistance. Moreover, such polyimide may be used as a substrate material for flexible electronics, as well as a coating material for electrodes and other components;
(ii) Elastomers: Elastomers such as polydimethylsiloxane (PDMS), polyurethane, and silicone may be used in flexible bioelectronic devices due to their softness and stretchability. They may be used as substrate materials or as encapsulating materials for sensitive components such as sensors;
(iii) Conductive polymers: Conductive polymers such as poly(3,4-ethylenedioxythiophene) (PEDOT) and polypyrrole may be used as electrode materials in flexible bioelectronic devices. They have good electrical conductivity and may be deposited onto flexible substrates using various techniques such as inkjet printing and vapor deposition;
(iv) Carbon-based materials: Carbon-based materials such as graphene and carbon nanotubes have unique mechanical and electrical properties that make them suitable for use in flexible bioelectronic devices. They may be used as conductive materials, sensing materials, or even as structural materials due to their high strength-to-weight ratio;
(v) Organic semiconductors: Organic semiconductors such as pentacene and rubrene may be used in flexible bioelectronic devices to create flexible transistors, photovoltaics, and sensors. They may be deposited onto flexible substrates using various techniques such as spin coating and vapor deposition; and
(vi) Biodegradable materials: Biodegradable materials such as polylactic acid (PLA), polycaprolactone (PCL), and polyhydroxyalkanoates (PHA) may be used to create flexible bioelectronic devices that are designed to degrade over time. Flexible bioelectronic devices may be useful for temporary implantation or for applications where biocompatibility is a critical concern.

1. An implantable bioelectronic device (100, 300, 1102), the implantable bioelectronic device comprising
a base material (102) with a top layer (102A) and a bottom layer (102B) opposite the top layer, the base material comprising at least one electrical component (104); and
a biological sample (108, 202, 1106) seeded on the top layer of the base material.

2. The implantable bioelectronic device (100, 300, 1102) of claim 1, wherein the base material (102) has a Young's modulus measurement of stiffness no greater than 1 GPa to mimic the Young's modulus measurement of stiffness of the target tissue or organ, wherein the target tissue or organ includes nervous system tissue or the brain.

3. The implantable bioelectronic device (100, 300, 1102) of claim 1, when in use in vivo, enables connecting a first element (114) and a second element (116) for restoration of an interrupted biological function between the first and second elements.

4. The implantable bioelectronic device (100, 300, 1102) of claim 1, wherein the biological sample (108, 202, 1106) can be seeded on a hydrogel (110, 1108) that includes at least one of: a fibrin hydrogel, a poly(ethylene glycol) (PEG) hydrogel, a poly(acrylic acid) (PAA) hydrogel, an alginate hydrogel, a chitosan hydrogel, a gelatin-based hydrogel.

5. The implantable bioelectronic device (100, 300, 1102) of claim 1, wherein the base material (102) includes at least one: Polyimide, elastomers, polydimethylsiloxane (PDMS), polyurethane, conductive silicone, polymers, poly(3,4-ethylenedioxythiophene) (PEDOT), polypyrrole, Carbon-based materials, graphene, Carbon nanotubes, organic semiconductors, pentacene, rubrene, biodegradable materials, polylactic acid (PLA), polycaprolactone (PCL), polyhydroxyalkanoates (PHA).

6. The implantable bioelectronic device (100, 300, 1102) of claim 1, wherein the implantable bioelectronic device is configured to record the restoration of the interrupted biological function between the first element (114) and the second element (116) as stimulation data.

7. The implantable bioelectronic device (100, 300, 1102) of claim 6, wherein implantable bioelectronic device (100, 300, 1102) is configured to provide the electrical stimulation as a pulse of an activation threshold ranging from 10 to 200 microampere using a pre-defined duration pulse.

8. The implantable bioelectronic device (100, 300, 1102) of claim 1, wherein the electrical component (104) includes at least two electrical components, and wherein the at least two electrical components are arranged in a symmetrical array occupying an area in a range of 1.0×1.0 millimetre to 10×10 millimetres within the base material (102).

9. The implantable bioelectronic device (100, 300, 1102) of claim 1, wherein the base material (102) comprises a polymer layer, selected from a parylene derivative, deposited on a flexible wafer, selected from a silicon, a glass, or polymers.

10. The implantable bioelectronic device (100, 300, 1102) of claim 1, wherein the biological sample (108, 202, 1106) is selected from an undifferentiated biological cell type such as human induced pluripotent stem cells (iPSC).

11. The implantable bioelectronic device (100, 300, 1102) of claim 3, wherein the first element (114) is an electrically active cell and the second element (116) is selected from an electrically active cell, muscle tissue and an electrical component.

12. The implantable bioelectronic device (100, 300, 1102) of claim 1, further comprising:
a processing arrangement for processing and analysing recorded stimulation data;
a memory unit;
a transmitter that is configured to translate the stimulation data, and
a battery unit.

13. A method of using an implantable bioelectronic device (100, 300, 1102) into a body of a subject of claim 1, the method comprising
performing an in vitro activity for cell culture on the implantable bioelectronic device, the in vitro activity comprising:
obtaining the implantable bioelectronic device,
seeding a biological sample (108, 202, 1106) on top of the implantable bioelectronic device and allowing the biological sample to grow for a pre-defined time; and
performing an in vivo activity comprising:
implanting the implantable bioelectronic device with the biological sample thereon into the subject at a desired location, wherein the implantation of the implantable bioelectronic device enables connecting a first element (114) and a second element (116) for restoration of an interrupted biological function between the first and second elements.

14. The method of claim 13, wherein the method includes implanting the implantable bioelectronic device (100, 300, 1102) in the subject such that a bottom layer of the implantable bioelectronic device is laid against a first part of the subject's body and a top layer having the biological sample (108, 202, 1106) thereon faces an electrically active cell proximal to the first part of the subject's body.

15. The method of claim 13, further comprising recording a stimulation data, in vivo, by the implantable bioelectronic device (100, 300, 1102).

16. The method of claim 13, wherein the method further includes providing the electrical stimulation as a pulse of an activation threshold ranging from 10 to 200 microampere using a pre-defined duration pulse.

17. The method of claim 13, wherein the number of electrical components (104) used is at least two, and wherein the at least two electrical components are arranged in a symmetrical array occupying an area in a range of 1.0×1.0 millimetre to 10×10 millimetre within the base material (102).

18. The method of claim 13, further comprising:
processing and analysing, using a processing arrangement, recorded stimulation data;
storing, in a memory unit, the recorded stimulation data;
translating, using a transmitter, the recorded stimulation data, and
powering, using a battery unit, the implantable bioelectronic device.

19. The method of claim 13, further comprising preparing the implantable bioelectronic device (100, 300, 1102) using at least one of: a photolithography technique, printing technique, and a metal lift-off technique.

20. A computer program product comprising a non-transitory machine-readable data storage medium having stored thereon program instructions that, when accessed by a processing arrangement, cause the processing arrangement to carry out the method claim 13.
